(19) United States
(12) Patent Application Publication
(10) Pub. No.: US 2007/0049909 A1
(43) Pub. Date: Mar. 1, 2007

(54) Magnetically enabled optical ablation device

(76) Inventor: Gareth T. Munger, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 11/508,739

(22) Filed: Aug. 23, 2006

Related U.S. Application Data
(60) Provisional application No. 60/711,776, filed on Aug. 26, 2005.

(51) Int. Cl. A61B 18/18

(52) U.S. Cl. 606/7

(57) ABSTRACT

A device for ablating material from a body lumen, the device including an elongate element having a longitudinal axis, the element having a proximal end, a distal end, and lumen therebetween; and means for delivering optical energy from the distal end of the elongate element at an angle with respect to the longitudinal axis of the element. The distal end of the device can include magnetically responsive elements so that the device can be oriented in an externally applied magnetic field.

### CROSS-REFERENCE TO RELATED APPLICATIONS

This application claims the benefit of U.S. Provisional Patent Application Ser. No. 60/711,776, filed Aug. 26, 2005, the entire disclosure of which is incorporated herein by reference.

### BACKGROUND

This invention relates to the removal of material from body lumens and cavities and in particular to the optical removal of obstructive material from blood vessels, such as partial blockages and chronic total occlusions, achieved by navigating an interventional device through a patient vasculature.

Various attempts have been made to provide for the removal of material from body lumens, such as blood vessels. For example, rotating burrs have been developed which can be navigated through the lumen to mechanically remove material forming blockages in the lumen. One example of such a device is disclosed in U.S. Pat. Nos. 6,740,103 and 6,733,511 for magnetically navigable and/or controllable device for removing material from body lumens and cavities, incorporated herein by reference. Other devices have attempted to use energy (for example radio-frequency (RF) energy or optical energy) to ablate the material forming blockages in the lumen. One problem with the adoption of ablative technologies for use in chronic total occlusion (CTO) therapy has been the inability to adequately navigate to the CTO and then clear a path through the CTO. The key to the use of interventional and ablative technologies is in the ability to plan the procedure, guide the minimally invasive surgery, monitor and control the progress of the intervention, and verify that the therapy objectives have been met. In particular, monitoring and controlling the ablative device during the intervention is necessary to remove the CTO without damaging the vascular wall.

Many of the laser ablation devices presently available on the market use a fiber bundle and have outer diameters ranging from about 0.9 mm to about 4.9 mm, the larger devices having an interior lumen that allows the device to be inserted over an already placed guidewire. Many occlusions, such as those affecting the coronary arteries, are located in relatively small vessels distant from the medical device insertion point. Reaching such lesions is facilitated by use of small diameter devices that are more maneuverable at branch points and within convoluted anatomy. However, incorporating any kind of conventional directional control in such small devices has been difficult; devices utilizing internal cabling systems or similar mechanical control mechanisms for remote operation of the distal end are typically too large for access to small arteries. Another difficulty with at least some of the available devices is that the devices typically only clear an opening through a lumen with a dimension equivalent to the diameter dimension of the device, which must necessarily be small in order to reach occlusions in small arteries. Still another difficulty with at least some of the devices has been ablatively operating the devices in curved lumens. The devices tend to clear a path extending beyond the distal end and aligned with the axis of the device at the distal end, which makes it difficult to advance a device along a curved path. These two later difficulties compound: the path cleared by the device through an occlusion, being not significantly larger than the device diameter, precludes reorientation of the device tip to follow the curvature of the vessel within which the occlusion is lodged. These and other difficulties have limited the usefulness of ablative devices for clearing body lumens and vessels.

### SUMMARY

The present invention discloses ablative devices that address a number of the problems encountered with application of the currently available designs. In one embodiment a device is provided that provides ablative energy at an angle relative to the longitudinal axis of the device. This permits the device to clear a path through a curved lumen. Through operation of the ablative energy concurrently with rotation of the device distal end, such a design also permits clearing a path that is at least somewhat larger than the device itself. A number of embodiments of optical ablation devices are disclosed wherein magnetically responsive elements can be provided to allow the distal end to be oriented or positioned with an externally applied magnetic field. Through the magnetic forces and torques exerted on the device distal end, magnetic navigation enables easier progression of the therapy device to the blockage and improved maneuverability of the device in clearing a path through the occlusion.

A first embodiment of a device for ablating material from a body lumen in accordance with the principles of this invention comprises an elongate element having a longitudinal axis and an essentially circular cross-section. The element has a proximal end, a distal end, and lumen therebetween. The device further comprises means for delivering optical energy from the distal end of the elongate element at an angle with respect to the longitudinal axis of the distal end. This means could be at least one, and preferably a bundle, of optical fibers. These optical fibers may be bent adjacent the distal end at an angle or multiplicity of angles with respect to the longitudinal axis of the elongate element to direct optical energy from the distal end of the device at an angle or angles with respect to the longitudinal axis. Alternatively, the distal ends of the optical fibers can be beveled and optically coupled to a lens for delivering the optical energy at an angle with respect to the longitudinal axis of the device; in yet a further embodiment, groups of one or more optical fibers can be beveled and each group optically coupled to one of a multiplicity of lenses for delivering optical energy at a range of angles with respect to the device longitudinal axis.

In accordance with several embodiments of this invention, magnetically responsive elements can be provided to allow the distal end of the device to be oriented using an externally applied magnetic field. These magnetically responsive elements can comprise permanent or permeable magnetic material and/or electromagnetic coils. The orientation of the distal end of the device, and thus the direction of the delivery of the optical energy can be controlled by controlling the direction of the applied magnetic field, and by controlling the rotation of the device with respect to the longitudinal axis through torques applied at the device proximal end.

Thus, devices of the various embodiments of this invention provide ablative energy at an angle or at a multiplicity of angles relative to the longitudinal axis of the device, permitting the device to clear a path through a curved lumen. Through rotation of the distal end with respect to its longitudinal axis, as induced for instance by mechanical torques applied at the device proximal end, the device can clear a path that is at least somewhat larger than the device itself. These and other embodiments and advantages of the present invention will become apparent from the following detailed description, and the accompanying drawings, which illustrate by way of example the features of the invention.

### BRIEF DESCRIPTION OF THE DRAWINGS

FIG. 1 is a schematic diagram of a first embodiment of an ablation device for removing material from body lumens, in accordance with the principles of this invention.

FIG. 2 presents operation of the device of FIG. 1 in a bent and occluded vessel.

FIG. 3 shows operation of the device of FIG. 1 at an occluded vessel branch.

FIG. 4 illustrates clearing of a path larger than the device diameter through application of ablative optical energy to the device of FIG. 1 concurrently with device rotation with respect to its distal end longitudinal axis.

FIG. 5 presents an alternative embodiment of the present invention comprising beveled optical fibers and an optical lens for the redirection of optical energy at angle with respect to the device distal end longitudinal axis.

FIG. 6 shows one embodiment of the device according to the principles of the present invention comprising a magnetic sleeve or ring tip with a hollow center providing a lumen for passage of several optical fibers and further comprising a hollow core that is suitable for insertion over a guide wire.

FIG. 7 presents cross-sections for a first set of three possible distal end designs including a magnetic element.

FIG. 8 presents cross-sections for a second set of three possible distal end designs including a magnetic element.

FIG. 9 presents cross-sections for a third set of three possible distal end designs including a magnetic element.

Corresponding reference numerals indicate corresponding parts throughout the several views of the drawings.

### DETAILED DESCRIPTION

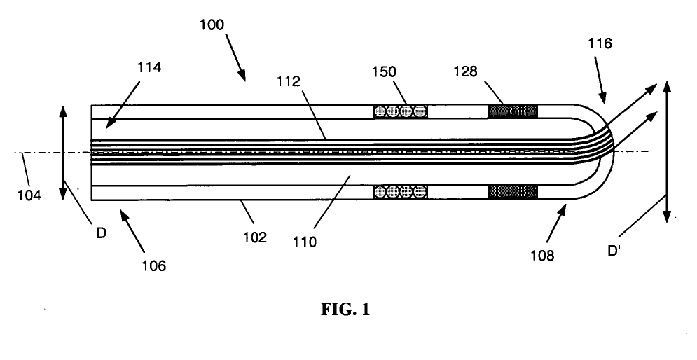

A first embodiment of a device for ablating material from a body lumen is indicated generally as 100 in FIG. 1. Generally, the device 100 comprises an elongate element with means for delivering optical energy from the distal end of the elongate element at an angle with respect to the longitudinal axis of the element.

More specifically, and as shown in FIG. 1, the device comprises an elongate element 102 having a longitudinal axis 104. The element has a diameter D, defining a cross sectional area and corresponding circumference. The element 102 has a proximal end 106, a distal end 108, and lumen 110 therebetween. In this first preferred embodiment at least one optical fiber, and preferably a bundle 112 of optical fibers, having a proximal end 114 and a distal end 116, extend from the device proximal end 106 substantially to the distal end 108. The distal end 116 of the fiber optical bundle 112 is oriented at an angle with respect to the longitudinal axis 104 of the element 102, to direct optical energy delivered to the proximal end of the fiber optic bundle from the distal end of the device and at angle with respect to longitudinal axis to ablate material beyond the circumference of the device. As shown in FIG. 1, in the preferred embodiment the distal end portion of the fiber optic bundle 112 bends to an angle of at least 20° with respect to the longitudinal axis of the element, and more preferably at an angle of at least 35° with respect to the longitudinal axis of the element. The ablative range of the delivered optical energy is from one to several hundred microns, depending on applied power, light wavelengths, and surrounding materials.

The device 100 preferably also includes at least one magnetically responsive element adjacent the distal end of the element for orienting the distal end in an applied magnetic field of about 0.1 Tesla, and more preferably in an applied magnetic field of about 0.08 Tesla, and still more preferably in an applied magnetic field of about 0.06 Tesla. The one or more magnetically responsive elements can comprise magnetic bodies, such as magnetic rings 128, which can be made of a permanent magnetic material such as neodymium-iron-boron (Nd—Fe—B), but could alternatively comprise a permeable magnetic material such as Hiperco. Alternatively, or in addition, the magnetically responsive element could comprise an electromagnetic coil 150.

In use, the device 100 is connected to a source of optical energy, such as a laser. Optical energy is conducted by the fiber optic bundle 112 from the proximal end 114 to the distal end 116. Because of the bend in the fiber optic bundle, the optical energy emanates from the distal end of the device at an angle with respect to the longitudinal axis of the device. The magnetically responsive elements help to orient the device so that it can be navigated through the body lumen, and in particular help to navigate the device through bends and branches in the body lumen, and to control the orientation of the device to clear a wide path through the body lumen.

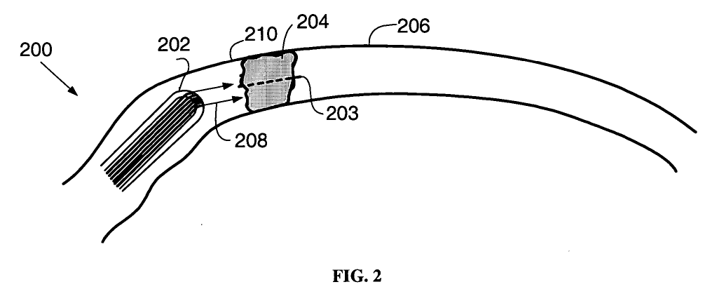

As illustrated in FIG. 2, this facilitates clearing a path through a bending lumen, more so than a device that has a straight fiber optic end aligned with the longitudinal axis of the device. FIG. 2 illustrates use of the device of FIG. 1 in clearing a path through a bent and obstructed vessel, 200. Through navigation, facilitated by magnetic orientation of the distal end, the device tip 202 is now adjacent to the occlusion 204. The device orientation with respect to the vessel 206 is such that the emitted light 208 is essentially oriented parallel to the local vessel axis 203 through the occlusion 204. In such a manner, a path can be cleared through the bent vessel occlusion with significantly reduced risk of injury to the vessel wall 210.

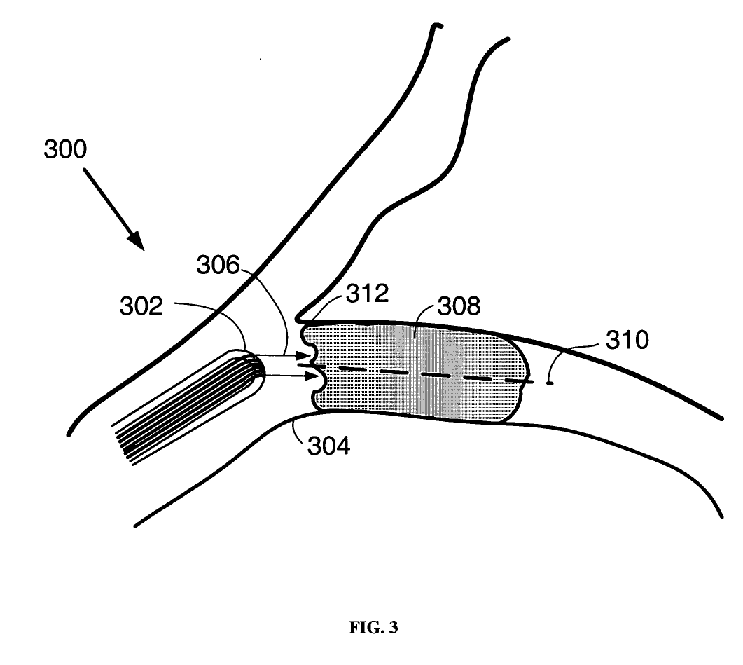

FIG. 3 generally shows 300 using the device of FIG. 1 to clear an occlusion located at a vessel branch. Through navigation and application of a torque at the device proximal end, the device tip 302 has been positioned and oriented with respect to vessel branch 304 such that emitted light 306 is substantially parallel to the vessel occlusion 308 local longitudinal axis 310. Use of a device that emits ablative radiation at an angle with respect to the device axis reduces the risk of injury to the vessel wall 312.

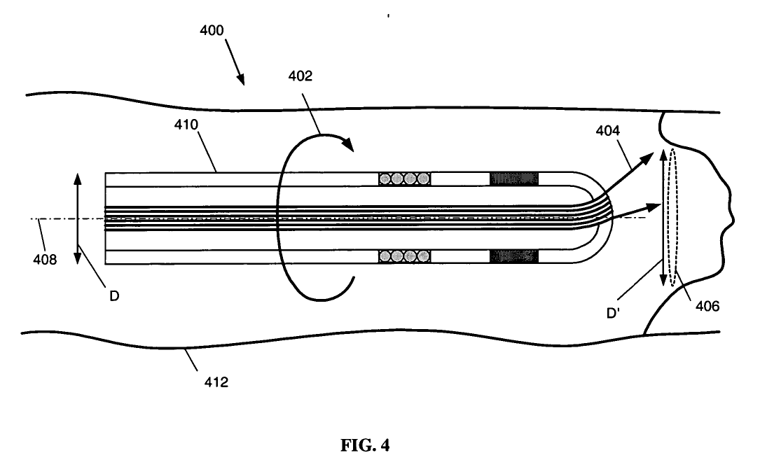

Moreover, if the device is rotated, it is possible to clear a path larger than the device itself. For example, and as illustrated generally by 400, FIG. 4, and depending upon the power and wave length of the optical energy provided to the device, rotating the device clears a path of diameter D′, which is larger that the diameter D of the device. Upon rotation 402, the beam 404 describes a cone of base 406. As the beam 404 is angled with respect to the device distal end longitudinal axis 408, the cone base 406 diameter is larger than the device tip 410 diameter.

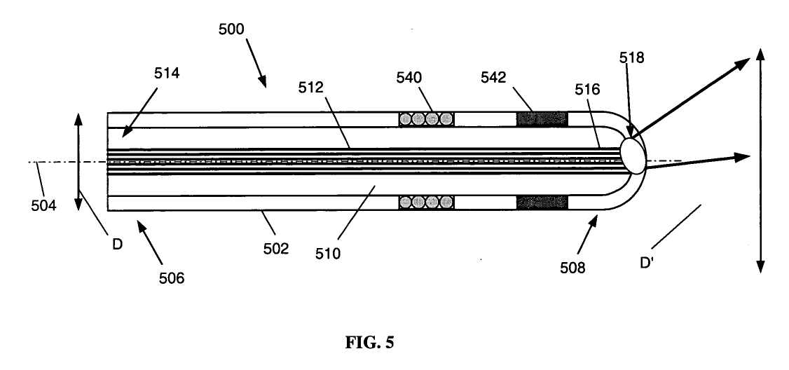

A second embodiment of a device for ablating material from a body lumen is indicated generally as 500 in FIG. 5. Generally, the device 500 comprises an elongate element with means for delivering optical energy from the distal end of the elongate element at an angle with respect to the longitudinal axis of the element. More specifically, and as shown in FIG. 5, the device comprises an elongate element 502 having a longitudinal axis 504. The element has a diameter D, defining a cross sectional area and corresponding circumference. The element 502 has a proximal end 506, a distal end 508, and lumen 510 therebetween. In this second preferred embodiment at least one optical fiber, and preferably a bundle 512 of optical fibers, having a proximal end 514 and a distal end 516, extend from the device proximal end 506 substantially to the distal end 508. The distal end 516 of the fiber optical bundle 512 preferably has a beveled face oriented at an angle with respect to the longitudinal axis 504 of the element 502. A lens 518 is optically coupled to the distal end of the fiber bundle, to direct optical energy delivered to the distal end of the fiber optic bundle at angle with respect to the longitudinal axis to ablate material beyond the circumference of the device. As shown in FIG. 5, in the preferred embodiment the lens 518 delivers optical energy at an angle of at least 20° with respect to the longitudinal axis of the element, and more preferably at an angle of at least 35° with respect to the longitudinal axis of the element.

The device 500, like device 100, preferably also includes at least one magnetically responsive element 540 adjacent the distal end of the element for orienting the distal end of the element in an applied magnetic field of about 0.1 Tesla, and more preferably in an applied magnetic field of about 0.08 Tesla, and still more preferably in an applied magnetic field of about 0.06 Tesla. The one or more magnetically responsive elements can comprise magnetic bodies, such as magnetic rings 542, which can be made of a permanent magnetic material such as neodymium-iron-boron (Nd—Fe—B), but could alternatively comprise a permeable magnetic material such as Hiperco. Alternatively, or in addition, the magnetically responsive element could comprise an electromagnetic coil.

In use, the device 500 is connected to a source of optical energy, such as a laser. Optical energy is conducted by the fiber optic bundle 512 from the proximal end 514 to the distal end 516. Because of the lens 518, the optical energy emanates from the distal end of the device at an angle with respect to the longitudinal axis of the device. This facilitates clearing a path through a bending lumen, more so than a device that has a straight fiber optic end aligned with the longitudinal axis of the device. Moreover, if the device 500 is rotated, it is possible to clear a path opening within a vessel larger than the device itself, as illustrated in FIG. 4 for a previously described device. The magnetically responsive elements help to orient the device so that it can be navigated through the body lumen, and in particular help to navigate the device through bends and branches in the body lumen, and to control the orientation of the device to clear a wide path through the body lumen.

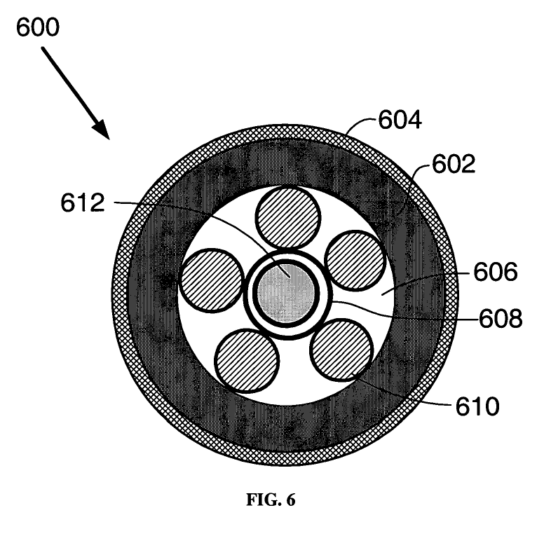

FIG. 6 generally shows a cross-section 600 of a device distal end designed according to the principles of this invention. In the embodiment of FIG. 6, the device tip, extending over a few millimeters along the device longitudinal axis, comprises a hollow cylindrical magnet element 602 made of either a permanent magnet material or a permeable material. In one embodiment the device also comprises an external cladding layer 604. A hollow cylindrical opening 606 is formed into the magnetic material to provide passage for optical fibers 610 as well as an inner tubing element 608 made, for example, of a polymer material. Element 608 provides a lumen through the device tip and substantially through the device length to allow the device to be inserted over a guide wire 612. In the geometry of FIG. 6, the outer diameter of the magnet element 602 is preferably less than 3 mm, and more preferably less than 2 mm.

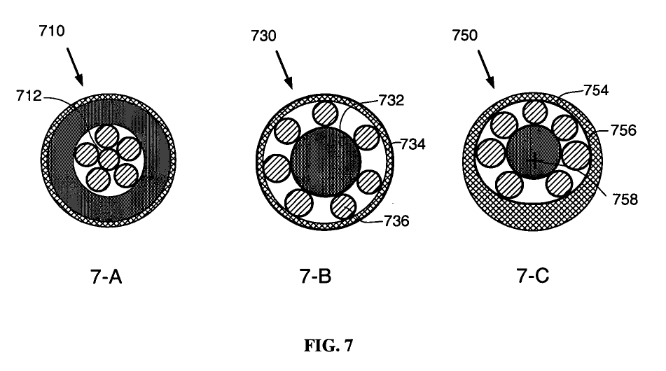

FIG. 7 illustrates three alternate embodiments of the device tip shown in cross-section. The embodiment of FIG. 7-A, 710, is generally similar to that of FIG. 6; however the cylindrical opening is completely filled by the fiber optics bundles 712. In the embodiment illustrated in FIG. 7-B, the magnet element 732 is cylindrical and enclosed in a tubular structure 734 which encloses a multiplicity of optical fibers 736. The alternate embodiment of FIG. 7-C is similar to that of FIG. 7-B, but the outer tubular element 754 has an offset internal circular cross-section 756, such that the fiber optics are not equally distributed with respect to the device longitudinal axis 758 but rather off-centered.

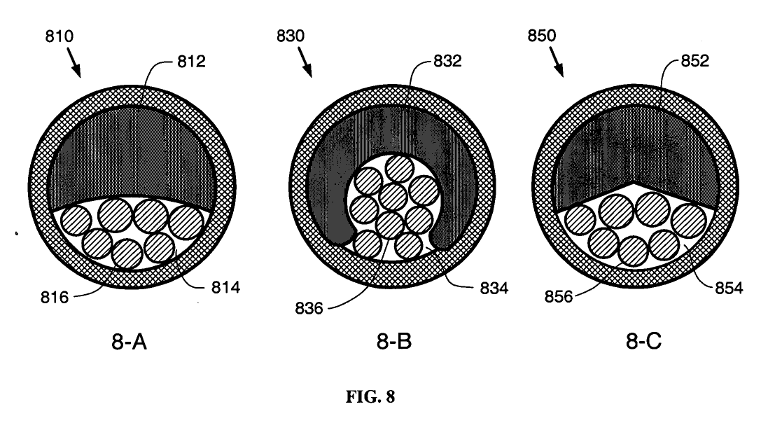

FIG. 8 shows three alternate embodiments in cross-section. In these three embodiments generally illustrated by 810, 830, and 850, an inner magnet element is shaped to occupy part of the volume within an outer tubular element. The optical fibers occupy the remaining volume internal to the outer tubular element. In FIG. 8-A, the magnet cross-section 812 assumes a filled “D” shape; the fibers 814 are distributed in the area 816. In FIG. 8-B, the magnet cross-section takes the shape of an extended half-moon 832. Finally, in FIG. 8-C, the magnet cross-section 852 is pie-shaped.

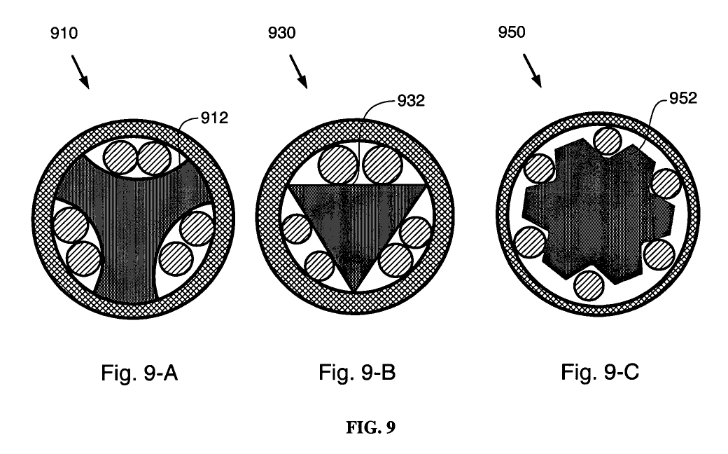

FIG. 9 illustrates three alternate embodiments in cross-section. In the embodiments of FIG. 9-A, 910, 9-B, 930, and 9-C, 950, respectively, the magnet element assumes the cross-section shape of a circular section with three cut-outs, 912, a triangular cross-section, 932, and a multi-faceted shape with multiple notches, 952.

Various embodiments offer trade-offs between ease of manufacturability, magnitude of the resulting magnetic moment, and ease of operation.

In accordance with a preferred embodiment of a method of ablating material from a body lumen to form a passage therein in accordance with the principles of this invention, a device such as device 100 or device 500 is disclosed comprising an elongate element having a longitudinal axis, and a fiber optic bundle for delivering optical energy from the distal end of the device at an angle with respect to the longitudinal axis of the device. The device can be navigated through a body lumen such as a blood vessel, by applying a magnetic field from an external source to orient the device and then mechanically advancing the device in the desired direction. Once at the site of a blockage, optical energy can be provided to the proximal end of the fiber optic bundle, which conducts the energy through the device and out the distal end of the device at an angle with respect to the longitudinal axis to ablate material blocking the vessel. The emitted radiation forms a path at an angle or angles with respect to the current orientation of the device, facilitating navigating the device through a bend or a branch in the blood vessel. The distal end of the device can be oriented in the direction of the cleared path, for example by applying the appropriate magnetic field from an external source magnet, and the device advanced through the bend or branch by repeating these steps. The device can also be rotated about its longitudinal axis, so that the path formed by the ablative beam sweeps a cone extending from the distal end of the device, clearing an area larger than the cross sectional area of the device.

Although the present invention has been described with respect to several exemplary embodiments, there are many other variations of the above-described embodiments that will be apparent to those skilled in the art, even where elements have not explicitly been designated as exemplary. It is understood that these modifications are within the teaching of the present invention, which is to be limited only by the claims appended hereto.

What is claimed is:

1. A device for ablating material from a body lumen, the device comprising: an elongate element having a longitudinal axis, the element having a proximal end, a distal end, and lumen therebetween; and means for delivering optical energy from the distal end of the elongate element at an angle with respect to the longitudinal axis of the element.

2. A device for ablating material from a body lumen, the device comprising: an elongate element having a longitudinal axis, the element having a proximal end, a distal end, and lumen therebetween; a fiber optic bundle having a proximal end and a distal end, the fiber optic bundle extending from the device proximal end substantially to the device distal end, the distal end of the fiber optic bundle being oriented at an angle with respect to the longitudinal axis of the device to direct optical energy delivered from the distal end of the fiber optic bundle at an angle with respect to the device longitudinal axis.

3. A device for ablating material from a body lumen, the device comprising: an elongate element having a longitudinal axis, the element having a proximal end, a distal end, and lumen therebetween; a plurality of fiber optic bundles each having a proximal end and a distal end, the fiber optic bundles extending from the device proximal end substantially to the device distal end, the distal ends of the fiber optic bundles being oriented at angles with respect to the longitudinal axis of the device to direct optical energy delivered from the distal end of the fiber optic bundle at angles with respect to the device longitudinal axis.

4. The device according to claim 2 wherein the distal end portion of the fiber optic bundle bends to an angle of at least 20 degrees with respect to the longitudinal axis of the element.

5. The device according to claim 2 wherein the distal end of the fiber optic bundle bends to an angle of at least 35 degrees with respect to the longitudinal axis of the element.

6. The device according to claim 2 wherein the distal end of the fiber optic bundle is beveled, and further comprising a lens optically coupled to the distal beveled end to direct optical energy at an angle with respect to the longitudinal axis of the element.

7. The device according to claim 3 wherein at least one of the distal ends of the fiber optic bundles is beveled, and further comprising at least one lens coupled to the distal beveled ends to direct optical energy at angles with respect to the longitudinal axis of the element.

8. The device according to claim 2 further comprising at least one magnetically responsive element adjacent the distal end of the element for orienting the distal end of the element in an applied magnetic field of 0.1 Tesla or more.

9. The device according to claim 2 further comprising at least one magnetically responsive element adjacent the distal end of the element for orienting the distal end of the element in an applied magnetic field of 0.08 Tesla or more.

10. The device according to claim 2 further comprising at least one magnetically responsive element adjacent the distal end of the element for orienting the distal end of the element in an applied magnetic field of 0.06 Tesla or more.

11. The device according to claim 8 wherein the at least one magnetically responsive element comprises a permanent magnetic material.

12. The device according to claim 8 wherein the at least one magnetically responsive element comprises a permeable magnetic material.

13. The device according to claim 8 wherein the at least one magnetically responsive element is an electromagnetic coil.

14. The device according to claim 8 wherein the at least one magnetically responsive element comprises permanent magnetic material and an electromagnetic coil.

15. The device according to claim 8 wherein the at least one magnetically responsive element comprises permeable magnetic material and an electromagnetic coil.

16. A method of ablating material from a body lumen to form a passage therein, the method comprising:
(a) advancing a device, the device comprising: an elongate element having a longitudinal axis, the element having a proximal end, a distal end, and lumen therebetween; a fiber optic bundle having a proximal end and a distal end, the fiber optic bundle extending from the device proximal end substantially to the device distal end and directing optical energy from the distal end of the device at an angle relative to the longitudinal axis of the device; and
(b) providing optical energy to the proximal end of the fiber optic bundle;

17. The method of claim 16, further comprising rotating the device to ablate material in the body lumen and to open a passage through the lumen larger than the diameter of the device.

18. The method of claim 16, further comprising magnetically navigating the device through the patient vasculature.

19. The method of claim 16, further comprising magnetically navigating the device to position the device distal end with respect to an occlusion and rotating the device during optical irradiation.

20. The method of claim 16, further comprising (a) optically irradiating an occlusion; (b) magnetically advancing the device through the partial path cleared through optical irradiation; and repeating steps (a) and (b) as necessary to completely clear a path through the occlusion.

21. The method of claim 20, further comprising applying a torque at the proximal device end during optical irradiation to clear a path larger than the device distal diameter.

22. A method of ablating material from a body lumen to form a passage therein, the method comprising:
(a) advancing a device the device comprising: an elongate element having a longitudinal axis, the element having a proximal end, a distal end, and lumen therebetween; a fiber optic bundle having a proximal end and a distal end, the fiber optic bundle extending from the device proximal end substantially to the device distal end, the distal end of the fiber optical bundle being oriented at an angle with respect to the longitudinal axis of the device to direct optical energy delivered from the distal end of the device at an angle with respect to the device longitudinal axis to ablate material beyond the circumference of the device;
(b) providing optical energy to the proximal end of the fiber optic bundle; and
(c) rotating the device to ablate material in the body lumen and open a passage through the lumen larger than the diameter of the device.
